(19) United States
(12) Patent Application Publication
(10) Pub. No.: US 2022/0386866 A1
(43) Pub. Date: Dec. 8, 2022

(54) Pupillometry Systems, Methods, and Devices

(71) Applicant: Andrew Elliott Neice, Lake Oswego, OR (US)

(72) Inventor: Andrew Elliott Neice, Lake Oswego, OR (US)

(21) Appl. No.: 17/341,390

(22) Filed: Jun. 8, 2021

(51) Int. Cl. A61B 3/11; A61B 3/14; A61B 3/00; G16H 30/40

(52) U.S. Cl. CPC A61B 3/112; A61B 3/14; A61B 3/0058; G16H 30/40

(57) ABSTRACT

Pupillometry systems for measuring one or more pupillary characteristics of a patient are shown and described. The puillometry systems include at least one camera for capturing image data of one or more pupils, at least one radiation source configured to project radiation to the one or more pupils, and a computer system in data communication with the at least one camera, the computer system having a processor and a non-transitory computer-readable storage medium. The non-transitory computer-readable storage medium includes computer-readable instructions for collecting and time stamping the image data, processing the raw image data in such a way as to bring the images into conformance with standardized image parameters, identifying and measuring the one or more pupils in the image data, processing the image data to produce measurement data of change in the one or more pupillary characteristics, calculating a standardized output of measurement data for the one or more pupillary characteristics, and providing a mechanism to share or store this information with other users via a computer network. Furthermore the pupillometry system has the capability to predict expected values of pupillary indices for a given patient and display to the user the relationship between the expected value and the measured value.

### BACKGROUND

The present disclosure relates generally to pupillometry systems, methods, and devices. In particular, pupillometry systems and methods that automatically reference and incorporate external data in order to improve the utility of pupillometric measurements, and to improve the user's ability to interpret pupillometric measurements.

Changes in the pupil can occur as a result of medications, drugs, and/or toxins (e.g., cough and cold medications, anticholinergic drugs, benzodiazepines, amphetamines, cocaine, lysergic acid diethylamide, marijuana, other narcotics, poisonous mushrooms, belladonna, chloroform, etc.). Pupil changes caused by medications, drugs and toxins are generally temporary. In addition to causing changes in size and response, drugs and/or toxins may affect a characteristic known as pupillary unrest, which is the variation in pupil size about its mean. Determining pupil size, pupillary unrest, pupillary response, and/or other pupillary characteristics can be useful in determining the presence and/or type of medications drugs, and/or toxins in the system of a patient. Further, changes in pupil size and unequal pupil size can be indicators of other serious conditions such as head trauma, brain tumors, and/or stroke. Thus, measuring pupil size and responses can be important for appropriate treatment for a patient.

Devices that measure the radius, diameter, circumference, or other characteristics of a pupil are generally referred to as pupillometers, and the analysis of these measurements is referred to as pupillometry. Pupillometry has a wide variety of applications. For example, precise measurements of pupil size may be useful in planning surgery or other procedures on the eye. Abnormal changes in pupil size, or failure of pupil size to change in response to stimuli (i.e., fixed pupils) may indicate that the patient has a neurological injury. Changes in the pupil size with time (i.e., pupillary unrest) can be correlated with drowsiness. It may additionally correlate with consumption of certain pharmaceuticals, and pupillary unrest may be useful in determining the activity of these pharmaceuticals.

Examples of known pupillometers include pupillometers that use custom designed and configured cameras, lighting, image processing computing devices, displays and housings in order to capture and process images of the pupil. These pupillometers are single-purpose devices. Because these pupillometers tightly control all parameters of image capture they are able to assume constant lighting and capture conditions for any set of pupillographic data, and thus process this data in an identical way without modification.

Another approach uses a general purpose computing device coordinated with a general purpose image capture device, display screen, lighting, and possibly networking and other general purpose electronic or computing equipment. These pupillometers replicate the functions of pupillometers that rely on custom designed hardware but additionally have many of the capabilities and advantages of general purpose devices, and are frequently less costly. Pupillometers of this type have been deployed on a variety of platforms, including desktop computers paired with conventional cameras and lighting, as well as smartphone platforms.

Known pupillometers are not entirely satisfactory for the range of applications in which they are employed. Specifically, known pupillometers generally output raw data about the pupil for the clinician to interpret. For example, known pupillometers will generally report the measured diameter of the pupil in millimeters, or the ratio of the pupil diameter to the iris diameter, or pupillary unrest, or other characteristics of the pupil. These sorts of data are frequently referred to as “pupillary indices” and term that will be used in this document. These pupillary indices are generally presented as a number without any context as to the expected values for a patient. Unfortunately, for clinical applications, users typically are interested not in the absolute value of the parameter in question, but how it relates to the population average. For example, it is more valuable for a clinician to know that a patient's pupil diameter is smaller or larger than expected given the patient's age and other demographic characteristics, rather than the exact value of the diameter. This is true not only for pupil diameter, but for other pupillary characteristics such as pupillary unrest in ambient light.

Unfortunately, it is not possible for clinicians to reference expected values of pupillary characteristics, because the expected value of most pupillary characteristics does not exist in an absolute sense. For example, we can reference the expected height or weight of an adult male based on population studies, but unfortunately we cannot reference the expected pupil diameter of an adult male because it is highly dependent upon variables that may be poorly controlled. Specifically, the ambient light level, light spectral distribution, light location on the retina, and other measurement characteristics can markedly change the expected value. This is true for most pupillary indices, such as pupillary unrest in ambient light, noxious stimulus reflex, etc.

Thus the mean value of these indices is only relevant if we specify not only the index but the exact measurement conditions; specifically the expected value of most pupillary characteristics is dependent on features that will be dependent on the pupillometer in question. For example, one type of pupillometer may illuminate the pupil with a light of higher intensity than a second pupillometer. Because pupil diameter is dependent on light intensity (as well as many other things), the first pupillometer will typically give lower values of pupil diameter than the second one, all else being equal. This makes it very difficult for the clinician to interpret pupillary data correctly. Clinicians rely on the published medical literature to make treatment decisions, but because published studies may use different types of pupillometers, the clinicians cannot directly apply the findings of those studies to their patients because the output of pupillometers is not standardized between instruments.

Furthermore, it will be understood by one skilled in the art that even pupillometers that ostensibly have the same characteristics will show variation in measurement values due to subtle differences in construction. For example, two pupillometers that both illuminate the pupil with 100 lux of illumination may elicit different pupillary light reflexes in different patients due to differences in the exact placement of the lights, leading the radiation to fall on different parts of the retina that may vary in sensitivity to light. Other characteristics, such as image capture characteristics, may be superficially identical in different pupillometers but in practice yield different average values for pupillary characteristics.

In the above example, pupil diameter was used as an example of a pupil characteristic in need of standardization between measuring instruments, but it should be understood that many pupillary characteristics are dependent upon measurement conditions. For example, pupillary unrest, which has been shown to be useful for measuring opioid effect, is also dependent upon the level of lighting provided by the pupillometer, as well as the capture characteristics of the pupillometer, such as frame rate, resolution, and noise level. Thus pupillary unrest is another example of a pupillary characteristic that requires standardization between different types of pupillometer. It should be understood that many other pupil characteristics will give slightly different results on otherwise identical patients in identical clinical situations due to variation in lighting, variation in camera and capture characteristics, variation in image processing, and other variations.

In the above example, measurements of pupil characteristics varied depending on the exact capture parameters associated with the pupillometer, but it is understood that another cause of variation is due to the demographic characteristics of the patient. For example, as individuals grow, the size of the eyes and hence the expected pupil diameter changes as well. As another example, pupillary unrest is believed to be dependent upon parasympathetic tone, and likely decreases with advancing age. Thus, a clinician attempting to interpret data on pupil characteristics will need to adjust this information based on the demographic characteristics of the patient. Unfortunately, many clinicians may not be highly familiar with differences in pupil characteristics associated with demographic variables such as age and sex, and would be greatly assisted if the pupillometer automatically integrated information about the expected mean characteristics of the patient before displaying this information to the user.

Thus there exists a need for pupillometry systems, methods, and devices that address the inability of current pupillometers to display and store data for the user in a way that can be easily and accurately interpreted. Examples of new and useful pupillometry systems, methods, and devices relevant to addressing these shortcomings are discussed below.

Disclosure addressing one or more of the identified existing needs is provided in the detailed description below. Examples of references relevant to pupillometers include U.S. Patent References: U.S. Pat. Nos. 7,083,280, 9,414,745, 3,782,364, 5,784,145, 7,625,087, and 8,127,882. The complete disclosures of the above patents are herein incorporated by reference for all purposes.

### SUMMARY

The present disclosure is directed to pupillometry systems for measuring one or more pupillary characteristics of a patient. The puillometry systems include at least one camera for capturing image data of one or more pupils, at least one radiation source configured to project radiation to the one or more pupils, and a computer system in data communication with the at least one camera, the computer system having a processor and a non-transitory computer-readable storage medium. The non-transitory computer-readable storage medium includes computer-readable instructions for collecting and time stamping the image data, identifying and measuring the one or more pupils in the image data, processing the image data to produce measurement data of change in the one or more pupillary characteristics, and calculating a standardized output of measurement data for the one or more pupillary characteristics. The non-transitory computer-readable storage medium additionally includes computer-readable instructions for one or more of the following

Predicting the expected value for a given pupillary parameter based on characteristics intrinsic to the device, including, but not limited to, such characteristics as lighting, capture noise level, frame rate, and other characteristics. The expected value may be based upon population studies that were conducted separately, in which measurements were taken on study subjects, and the data from these studies are stored locally in the device, or the data may be retrieved from an outside source. Alternatively the expected value may be calculated based on empirical formulas that compensate for differences in capture parameters intrinsic to the pupillometer,

Predicting the expected variance (for example the standard deviation or some other measure of variance) for a given pupillary parameter based on characteristics intrinsic to the device, including, but not limited to, such characteristics as lighting, capture noise level, frame rate, and other characteristics. The expected variance may be based upon population studies that were conducted separately, in which measurements were taken on study subjects, and the data from these studies are stored locally in the device, or the data may be retrieved from an outside source. Alternatively the expected value may be calculated based on empirical formulas that compensate for differences in capture parameters intrinsic to the pupillometer.

Predicting the expected value for a given pupillary parameter based on characteristics of the patient being measured that have been input into the device, such as age, sex, or other demographic characteristics. The expected value may be based upon population studies that were conducted separately, in which measurements were taken on study subjects, and the data from these studies are stored locally in the device, or the data may be retrieved from an outside source. Alternatively the expected value may be calculated based on empirical formulas that compensate for differences in capture parameters intrinsic to the pupillometer.

Predicting the expected variance for a given pupillary parameter based on characteristics of the patient being measured that have been input into the device, such as age, sex, or other demographic characteristics. The expected variance may be based upon population studies that were conducted separately, in which measurements were taken on study subjects, and the data from these studies are stored locally in the device, or the data may be retrieved from an outside source. Alternatively the expected value may be calculated based on empirical formulas that compensate for differences in capture parameters intrinsic to the pupillometer.

Displaying to the user information on measured pupillary characteristics relative to the values expected, instead of or in addition to, the absolute values. For example, if the mean and standard deviation have been calculated, the device may display a percentile associated with this pupillary measurement. Alternatively, the device may display a Z score, a statistical tool well known to those skilled in the art, in which a Z score of zero corresponds to the mean, +1 to one standard deviation above the mean, −1 to one standard deviation below, etc., Alternatively some other intuitive scale may be used, in which some centering value is known to correspond to the population mean. Lastly, if a known range of adjusted pupillary characteristics is known to have some clinical relevance, the data may be annotated with special color coding or other images in order to assist the clinician in their interpretation.

Storing, transmitting, or re-displaying the adjusted data

### BRIEF DESCRIPTION OF THE DRAWINGS

FIG. 1 is a schematic view of an example of a programmable computing device.

FIG. 2 is a schematic view of a first example pupillometry system including a programmable computing device, such as the example device shown in FIG. 1.

FIG. 3 is a flow diagram of a first example method of the first example pupillometry system shown in FIG. 2

FIG. 4 is an example method of operations for capturing pupil data with the device, along with an example output screen that can be displayed on the display of the first example pupillometry system shown in FIG. 2.

### DETAILED DESCRIPTION

The disclosed pupillometry systems, methods, and devices become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various pupillometry systems, methods, and devices are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

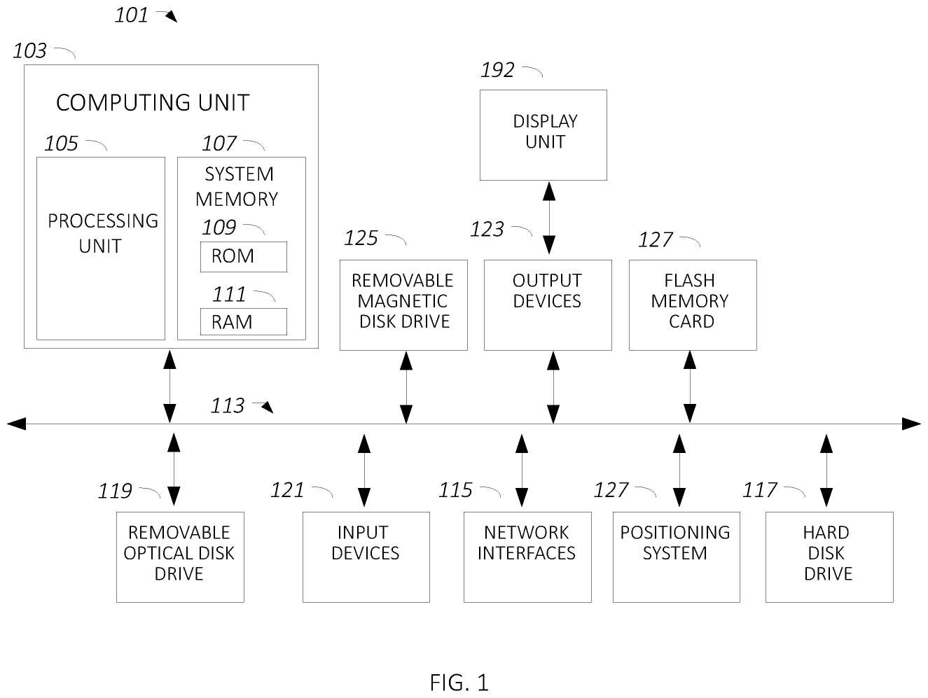

With reference to FIGS. 1-4, a first example of an enhanced pupillometry system, system 200, will now be described. Like existing pupillometry systems, pupillometry system 200 allows for an operator 202 to collect and analyze pupillometry data collected from a patient 204. Specifically, pupillometry system 200 functions to collect, process, and analyze puillometry data from patient 204 and display a standardized output of pupillometry data to operator 202. Additionally or alternatively, system 200 can be used to diagnose the patient or determine the presence and/or type of medications, drugs, or toxins that may have been consumed by the patient.

Like existing pupillometry systems, System 200 includes a computer system 206 having at least a non-transitory computer-readable storage medium 208, a processor 210, and a display 212. System 200 further includes a positioning device 214 for positioning a radiation source 216 and a camera 218 in a position to project light into and capture image data from one or more of the pupils of the patient. System 200 may optionally further includes a communication device 220 to transmit and receive data with a remote computer system 222 as well as a positioning system 224 that is able to determine the approximate location of the system on the earth's surface. Lastly, system 200 may optionally include an interface 226, by which the user can enter demographic information associated with the measurement subject 204; this interface may be integrated with the display 212 or may be a separate input device.

Pupillometry system 200 addresses many of the shortcomings existing with conventional pupillometers described in the background section. For example, the presently described system 200 can reference data stored in storage medium 208 average or expected values and variations for pupillary indices based on the properties of the specific pupillometer, and/or demographic data of the patient. The storage medium 208 may simply contain average or expected values and variations obtained by population studies using the specific pupillometer in question, or it may use predictive algorithms to determine the expected and average values and variations, or it may use some combination of data and algorithms.

In the preferred embodiment of this invention, the expected values and variances of pupillary indices are determined using a combination of empirically measured parameters and interpolation. Ideally, a large number of measurements on experimental subjects may be taken using an identical device prior to clinical deployment, and the average and standard deviation of the pupillary indices measured during this study may be stored in storage medium 208 for later retrieval. The measurements on experimental subjects may also include relevant demographic data that can be used in a linear regression or other predictive model to interpolate how the expected mean and standard deviation will change with age, sex, or other demographic characteristics. This allows a modified, more accurate mean and standard deviation to be supplied to the user when demographic data about the patient 204 is supplied to the system 202, although it is not strictly necessary if the demographic data is not entered. Because the subjects were measured using an identical device, all of the uncertainty related to the particular device characteristics (such as lighting and other capture characteristics) is accounted for prior to the generation of an expected mean and standard deviation.

In an alternate embodiment of this invention, the device characteristics such as light intensity, frequency distribution, and spatial distribution, of the pupillometry system 202 may be carefully measured and this data may be used in an existing mathematical model of pupil reactivity to predict the likely value of pupillary indices. It will be appreciated by one skilled in the art that this method has the advantage that there is no need to recruit a large number of experimental subjects and this may be easily implemented. However, it will also be appreciated by one skilled in the art that this method is likely to be less accurate that a direct empirical measurement of the pupillary characteristics.

Prior to capturing pupillary data and calculating a pupillary index, the pupillometry system may optionally prompt the user 202 to enter demographic information using the interface 226, or access this data using information previously stored on its own storage device 208, or retrieve this information from a remote storage area using the communication device 220. If this information is not available, the average or expected values and variances can be calculated solely based on the characteristics of the system 200, however if this information is available it may optionally be used to improved the accuracy of the expected values and variances, combining the known characteristics of system 200 and patient 204 as described in the previous paragraph.

After capturing pupillary data and calculating a pupillary index, and after calculating and/or retrieving the expected value and variance of the index as described above, the pupillometry system 200 will display to the user 202 using display 212 some combination of the calculated index, the expected value of the index, and the expected variance of the index. For example, the display 212 may show the measured value, the mean value in a similar population, and the standard deviation in a similar population. The data may also be further processed by calculating a percentile score using techniques from statistics that are well known to those skilled in the art. They may be further processed by displaying non-numerical information that summarizes the result for the user, for example “very low”, “low”, “normal”, “high”, “very high” may be used. Alternatively if some pupillary index is known to have a clinical significance when it deviates some degree from an expected value, this may be displayed, for example “opioid responsive”, or “opioid nonresponsive”. Lastly the text may be accompanied by images or may be colored in such a way that the clinician is alerted to the significance of the measurement.

Various disclosed examples may be implemented using electronic circuitry configured to perform one or more functions. For example, with some embodiments of the invention, the disclosed examples may be implemented using one or more application-specific integrated circuits (ASICs). More typically, however, components of various examples of the invention will be implemented using a programmable computing device executing firmware or software instructions (i.e., computer-readable instructions), or by some combination of purpose-specific electronic circuitry and firmware or software instructions (i.e., computer-readable instructions) executing on a programmable computing device.

Accordingly, FIG. 1 shows one illustrative example of a computer, computer 101, which can be used to implement various embodiments of the invention. Computer 101 may be incorporated within a variety of consumer electronic devices, such as personal media players, cellular phones, smart phones, personal data assistants, global positioning system devices, and the like.

As seen in this figure, computer 101 has a computing unit 103. Computing unit 103 typically includes a processing unit 105 and a system memory 107. Processing unit 105 may be any type of processing device for executing software instructions (i.e., computer-readable instructions), but will conventionally be a microprocessor device. System memory 107 may include both a read-only memory (ROM) 109 and a random access memory (RAM) 111. As will be appreciated by those of ordinary skill in the art, both read-only memory (ROM) 109 and random access memory (RAM) 111 may store software instructions to be executed by processing unit 105.

Processing unit 105 and system memory 107 are connected, either directly or indirectly, through a bus 113 or alternate communication structure to one or more peripheral devices. For example, processing unit 105 or system memory 107 may be directly or indirectly connected to additional memory storage, such as a hard disk drive 117, a removable optical disk drive 119, a removable magnetic disk drive 125, and a flash memory card 127. Processing unit 105 and system memory 107 also may be directly or indirectly connected to one or more input devices 121 and one or more output devices 123. Input devices 121 may include, for example, a keyboard, touch screen, a remote control pad, a pointing device (such as a mouse, touchpad, stylus, trackball, or joystick), a scanner, a camera or a microphone. Output devices 123 may include, for example, a monitor display, an integrated display, television, printer, stereo, or speakers.

Still further, computing unit 103 will be directly or indirectly connected to one or more network interfaces 115 for communicating with a network. This type of network interface 115 is also sometimes referred to as a network adapter or network interface card (NIC). Network interface 115 translates data and control signals from computing unit 103 into network messages according to one or more communication protocols, such as the Transmission Control Protocol (TCP), the Internet Protocol (IP), and the User Datagram Protocol (UDP). These protocols are well known in the art, and thus will not be discussed here in more detail. An interface 115 may employ any suitable connection agent for connecting to a network, including, for example, a wireless transceiver, a power line adapter, a modem, or an Ethernet connection.

Still further, computing unit 103 will be directly or indirectly connected to one or more devices for ascertaining user position 127. As is known in the art, this will frequently be based on the Global Positioning System or a comparable satellite navigational system.

It should be appreciated that, in addition to the input, output and storage peripheral devices specifically listed above, the computing device may be connected to a variety of other peripheral devices, including some that may perform input, output and storage functions, or some combination thereof.

Computer 101 may be connected to or otherwise include one or more other peripheral devices, such as a telephone. The telephone may be, for example, a wireless “smart phone,” such as those featuring the Android or iOS operating systems. As known in the art, this type of telephone communicates through a wireless network using radio frequency transmissions. In addition to simple communication functionality, a “smart phone” may also provide a user with one or more data management functions, such as sending, receiving and viewing electronic messages (e.g., electronic mail messages, SMS text messages, etc.), recording or playing back sound files, recording or playing back image files (e.g., still picture or moving video image files), viewing and editing files with text (e.g., Microsoft Word or Excel files, or Adobe Acrobat files), etc. Because of the data management capability of this type of telephone, a user may connect the telephone with computer 101 so that their data maintained may be synchronized.

Of course, still other peripheral devices may be included with or otherwise connected to a computer 101 of the type illustrated in FIG. 1, as is well known in the art. In some cases, a peripheral device may be permanently or semi-permanently connected to computing unit 103. For example, with many computers, computing unit 103, hard disk drive 117, removable optical disk drive 119 and a display are semi-permanently encased in a single housing.

Still other peripheral devices may be removably connected to computer 101, however. Computer 101 may include, for example, one or more communication ports through which a peripheral device can be connected to computing unit 103 (either directly or indirectly through bus 113). These communication ports may thus include a parallel bus port or a serial bus port, such as a serial bus port using the Universal Serial Bus (USB) standard or the IEEE 1394 High Speed Serial Bus standard (e.g., a Firewire port). Alternately or additionally, computer 101 may include a wireless data “port,” such as a Bluetooth® interface, a Wi-Fi interface, an infrared data port, or the like.

It should be appreciated that a computing device employed according to the various examples of the invention may include more components than computer 101 illustrated in FIG. 1, fewer components than computer 101, or a different combination of components than computer 101. Some implementations of the invention, for example, may employ one or more computing devices that are intended to have a very specific functionality, such as a digital music player or server computer. These computing devices may thus omit unnecessary peripherals, such as removable optical disk drive 119, printers, scanners, external hard drives, etc. Some implementations of the invention may alternately or additionally employ computing devices that are intended to be capable of a wide variety of functions, such as a smartphone, desktop or laptop personal computer. These computing devices may have any combination of peripheral devices or additional components as desired.

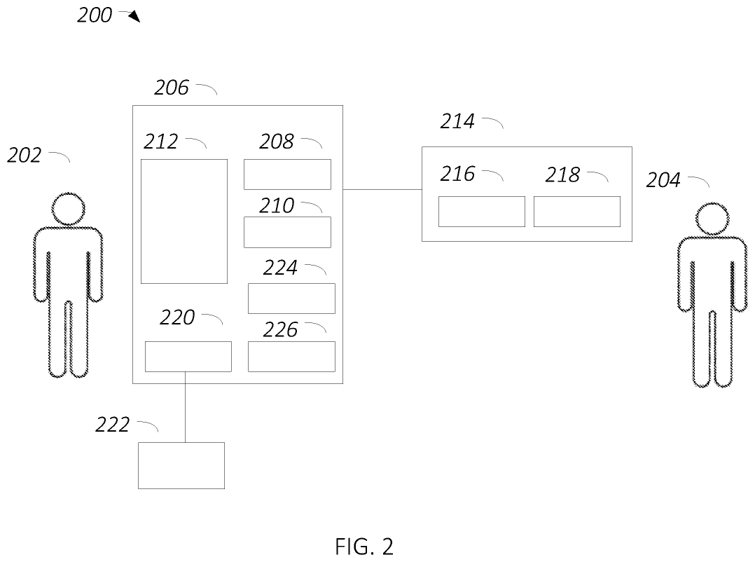

Turning now to FIG. 2, as described above, pupillometry system 200, computer system 206 (non-transitory computer-readable storage medium 208, processor 210, and display 212), positioning device 214 having radiation source 216 and camera 218, communication device 220, and positioning determining system 224. It will be understood that in alternate examples computer system 206 can include any of the additional computer system components described above in reference to computer 101 (shown in FIG. 1). It will be further understood that in other alternate examples the camera and the radiation source can have separate positioning mechanisms and/or can be hand-held.

In the preferred embodiment of this invention, the radiation source 216, camera 218, input, display, and other components of computing system 206 are all mounted together in a single housing, and the entire monolithic unit is hand held. In alternate examples, the camera and the radiation source(s) can be retained and/or mounted in a different positioning mechanism (e.g., mounted on a stand, mounted on a flexible arm, mounted in a pair of glasses, etc.).

Furthermore, in the present invention, if the operator 202 has entered into the system demographic data on the patient 204, operator 202 may tag the data with a unique identifier, or alternatively system 206 may automatically generate an identifier, and the data can then be stored locally on storage medium 208, or transferred to remote computer system 222 using communication device 220. Remote computer system 222 is accessible by multiple users. At a future date operator 202 may then receive this data from remote computer system 222 or from storage medium 208, so that the operator does not need to continually re-enter demographic data into the device.

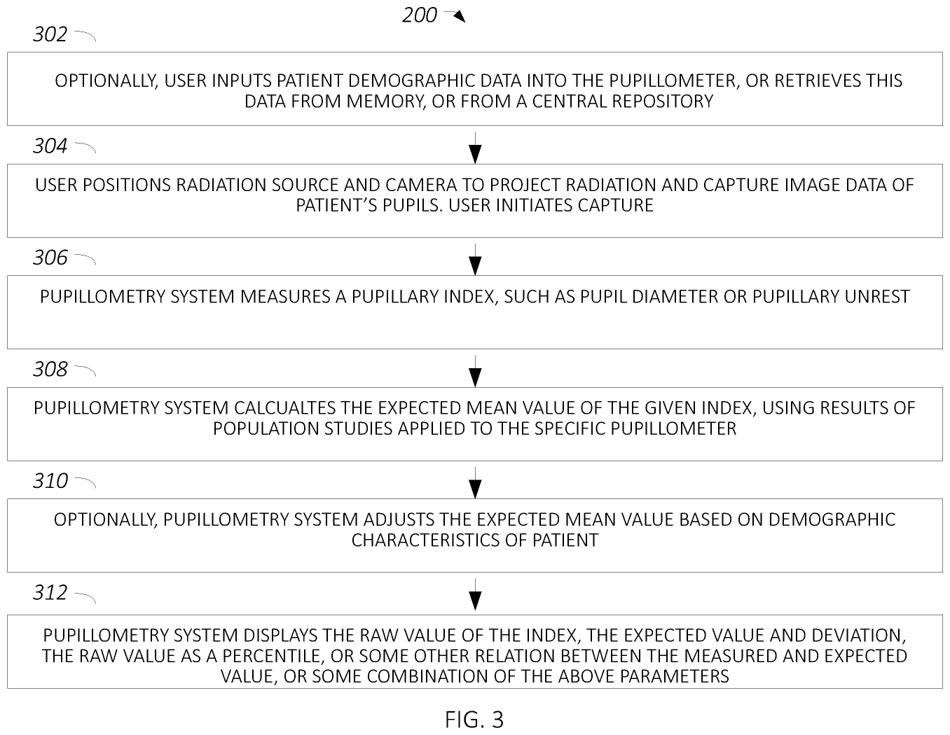

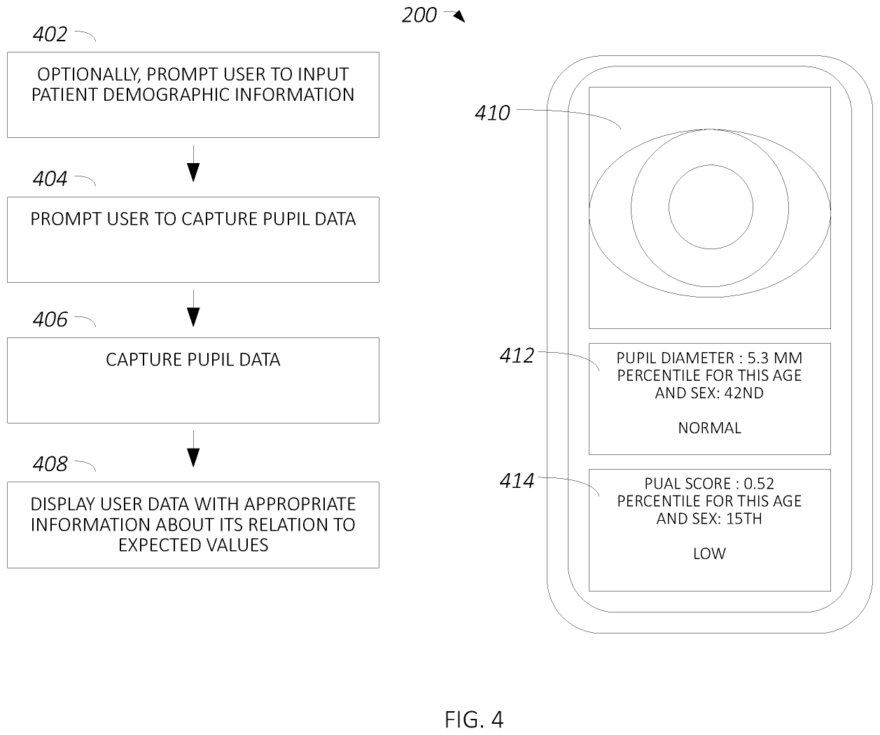

A partial example method for operating the pupillometry system, method 300, is shown in FIG. 3, which details the method for entering demographic data prior to beginning the process of data capture, and the process of retrieving this data and combining it with measured data to display to the user 202. Another partial example method of operations carried out by the computer system to improve the interpretation of pupillary indices after data capture, method 400, is shown in FIG. 4.

As depicted in FIG. 3, the user may optionally enter patient demographic data in step 302. In step 304-306 the system identifies and measures the pupil, and uses this to calculate a pupillary index, which may be as simple as simply the pupil diameter, or a more complex pupillary index such as pupillary unrest in ambient light, as described in other patents and scientific publications. In step 308, the pupillometry system 202 retrieves information regarding the expected value and variance of the index for this particular pupillometry system, and in step 310 it optionally modifies this expected value based on demographic characteristics entered for the patient 204. Finally, in step 312, the index measured and calculated in step 304-306 is displayed along with expected values and variances calculated in step 308-310.

An example method of operations for performing and displaying measurements using the above technique, along with the associated user display, is shown in FIG. 4. In this figure, during steps 402 the user is prompted to enter patient demographic data, and in steps 404-406 the user is prompted to center the camera on an individual's eye, which can be visualized on display 410. It should be obvious to one skilled in the art that although in this embodiment the user is prompted to enter demographic data prior to data capture, this step may take place after data capture. Furthermore it should be obvious to one skilled in the art that the demographic data may be retrieved from a local storage medium, or from a remote storage medium over a computer network.

The process of capturing data continues in a way common to a variety of existing pupillometers. The radiation source(s) is positioned to project radiation into one or both eyes of the patient and the camera is positioned to capture image data of the pupils from one or both eyes of the patient. Next, the operator sets and/or programs the computer system with the desired parameters for data collection (e.g., radiation pattern, timing for image capture, duration of data collection, pupillary characteristics to be measured, etc.). It will be appreciated that in alternate examples the parameters can be pre-set by the manufacturer (i.e., default settings). In these alternate examples, the operator can manually override and/or adjust the pre-set parameters. The operator then commands the computer system to begin data collection and analysis according to the desired parameters.

Apart from the input of patient demographic data, the preceding steps are common to a number of existing pupillometers. In the present invention, after data capture is complete a method is used to improve the user's ability to interpret the pupil data, by displaying the relationship between the measured pupil indices and the expected values. One embodiment of this method is shown in FIG. 4, in display sections 412 and 414. Here, in addition to displaying the raw value of the pupillary indices, the percentile score is calculated, which is calculating using the predicted mean and standard deviation of the pupillary indices of interest, which in turn is calculated using calibration data specific to the device in question as well as demographic characteristics of the patient.

Because different operators and/or healthcare providers may provide treatment for a single patient, and because changes in their pupil scans may be helpful in determining diagnosis or treatment plans, it may prove useful for an operator to share scans of a patient with other operators by communicating the scans or data associated with the scans to a central repository. This repository can be accessed by other authorized users, and in turn the operator can access their scans or retrieve his or her own scans. It should be appreciated by one skilled in the art, that if pupil scans are stored locally on the pupillometry system 202 or in a remote storage system using the communication device 220, in addition to the scans themselves the demographic information may also be stored, so that it does not need to be repeatedly re-entered.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinadons of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

### CITATION LIST

### Patent Literature

U.S. Pat. No. 9,414,745
U.S. Pat. No. 7,083,280
U.S. Pat. No. 3,782,364,
U.S. Pat. No. 5,784,145,
U.S. Pat. No. 7,625,087
U.S. Pat. No. 8,127,882

### Non-Patent Literature

AlgometRx pupillographic device, prototype description available athttps://atlanticpediatricdeviceconsortium.org/algometrx-pain-and-analgesic-drug-effect-measurement-device
NPi--200, Neuroptics Pupillometer, Product available at www.neuroptics.com.
McAnany, J. Jason, et al. “iPhone-based pupillometry: a novel approach for assessing the pupillary light reflex,” Optometry and Vision Science 95.10 (2018): 953. available athttps://www.ncbi.nlm.nih.gov/pmc/articles/PMC6166694/
De Souza, John Kennedy Schettino, et al., An open-source, FireWire camera-based, Labview-controlled image acquisition system for automated, dynamic pupillometry and blink detection, Computer Methods and Programs in Biomedicine, December 2013, pp. 607-623, vol. 112, Issue 3, available at http://www.sciencedirect.com/science/article/pii/S0169260713002460
Maz Warga et al., How do Spontaneous Pupillary Oscillations in Light Relate to Light Intensity?, Vision Research, February 2009, pp. 295-300, vol. 49, Issue 3, available at http://www.sciencedirect.com/science/article/pii/S0042698908004689
Anne-Claire et al., The Relevance of Pupillometry for Evaluation of Analgesia Before Noxious Procedures in the Intensive Care Unit. 2015, available athttps://www.ncbi.nlm.nih.gov/pubmed/25993266
B Wilhelm et al., Pupillographic Assessment of Sleepiness in Sleep-Deprived Healthy Subjects, Sleep 12.3, May 1998, pp. 258-265, available at http://www.pubfacts.com/detail/9595604/Pupillographic-assessment-of-sleep-Mess-in-sleep-deprived-healthy-subjects.

The invention claimed is:

1. A pupillometry system for measuring one or more pupillary characteristics of a patient, comprising: at least one camera for capturing image data of one or more pupils of the patient; at least one radiation source configured to project radiation to the one or more pupils; and a computer system in data communication with the at least one camera, the computer system including a processor and a non-transitory computer-readable storage medium, the non-transitory computer-readable storage medium having instructions for:
collecting and time stamping the image data,
identifying and measuring the one or more pupils in the image data,
processing the image data to produce measurement data of change in the one or more pupillary characteristics, and
calculating a standardized output of measurement data for the one or more pupillary characteristics
calculating an expected output of measurement data for the one or more pupillary characteristics, and
displaying the actual output, the expected output, and a standardized output of measurement of the relationship between the actual and expected output, or some combination thereof.

2. The pupillometry system of claim 1, wherein the expected output is based on experimental measurements on human subjects using a pupillometer having lighting and capture characteristics substantially similar to the pupillometry system.

3. The pupillometry system of claim 1, wherein the expected output is based on a predictive model that uses known lighting and capture characteristics of the pupillometry system to generate an expected index.

4. The pupillometry system of claim 1, wherein the expected output is further modified based on demographic characteristics including, but not limited to, age and sex.

5. The pupillometry system of claim 1, wherein the standardized output of measurement of the relationship between the actual and expected output is displayed as
the expected mean and standard deviation of the pupil index, and/or
a fraction or percentage of users that would be expected to have a value below the measured value, and/or
a fraction or percentage of users that would be expected to have a value above the measured value, and/or
a numerical scale in which a particular value is known to correspond to the expected measurement value, and deviations of the actual measurement from the expected measurement are expressed as deviations from this particular value, and/or
a word or image that corresponds to the fraction or percentage of users that would be expected to have a value above or below the measured value, and/or
coloration of text or images that corresponds to the fraction or percentage of users that would be expected to have a value above or below the measured value.

6. The pupillometry system of claim 1, wherein the pupillometry system has the capability to accept user input of demographic information of the subject relevant to the prediction of a pupillary index, and optionally store and retrieve this demographic information in a local or remote non-volatile storage medium.
